SEQUENCE LISTING (7 sequences)

SEQ ID NO: 1 - DNA, 19 bp, Artificial Sequence
  Synthetic upper primer to detect active form of ALDH2
aacgaagcccagcaaatga

SEQ ID NO: 2 - DNA, 20 bp, Artificial Sequence
  Synthetic lower-1 primer to detect active form of ALDH2
gggctgcaggcatacacaga

SEQ ID NO: 3 - DNA, 20 bp, Artificial Sequence
  Synthetic lower-2 primer to detect inactive form of ALDH2
gggctgcaggcatacacaaa

SEQ ID NO: 4 - DNA, 20 bp, Artificial Sequence
  Synthetic lower-1A primer to detect active form of ALDH2
gggctgcaggcatacacaga

SEQ ID NO: 5 - DNA, 20 bp, Artificial Sequence
  Synthetic lower-1C primer to detect active form of ALDH2
gggctgcaggcatacaccga

SEQ ID NO: 6 - DNA, 20 bp, Artificial Sequence
  Synthetic lower-2A primer to detect inactive form of ALDH2
gggctgcaggcatacacaaa

SEQ ID NO: 7 - DNA, 20 bp, Artificial Sequence
  Synthetic lower-2C primer to detect inactive form of ALDH2
gggctgcaggcatacaccaa

(19) United States
(12) Patent Application Publication
(10) Pub. No.: US 2004/0121374 A1
(43) Pub. Date: Jun. 24, 2004

(54) Method for detecting single nucleotide polymorphisms

(76) Inventors: Yoshihide Iwaki, Asaka-shi (JP); Yoshihide Makino, Asaka-shi (JP)

Correspondence Address: BIRCH STEWART KOLASCH & BIRCH, PO BOX 747, FALLS CHURCH, VA, 22040-0747, US

(21) Appl. No.: 10/674,387

(22) Filed: Oct. 1, 2003

(30) Foreign Application Priority Data
Oct. 2, 2002 (JP) 2002-289567
Oct. 2, 2002 (JP) 2002-289566

(51) Int. Cl. C12Q 1/68; C12P 19/34

(52) U.S. Cl. 435/6; 435/91.2

(57) ABSTRACT

The object of the invention is to provide a method for accurately, simply, and swiftly detecting single nucleotide polymorphisms. The present invention provides a method for detecting single nucleotide polymorphisms, which utilizes two types of allele-specific primers designed in such a way that the amounts of be amplified products of each of heterozygous alleles are substantially the same, as well as a method for detecting single nucleotide polymorphisms, which utilizes two types of allele-specific primers undo such polymerase reaction conditions that the amounts of the amplified products of each of heterozygous alleles are substantially the same.

### TECHNICAL FIELD

The present invention relates to the detection of a specific DNA sequence of a specimen, the detection of genetic polymorphisms, the analysis of single nucleotide polymorphisms (SNPs), and the like.

### BACKGROUND ART

Accomplishments of genomic analysis and research have been concentrated in the systematic analysis of genetic polymorphism and the systematic analysis of gene expression. Recently, attempts have actively been made in order to utilize these genome information in the field of medicine, and technologies for analyzing genetic polymorphisms and gene expression have made astonishing progress.

Among genetic polymorphisms, single nucleotide polymorphisms (SNPs) are considered to occur at a rate of 1 per 1,000 nucleotides. These SNPs are considered to cause individual differences, individual characteristics, or congenital and constitutional differences. In addition, a factor gene is associated as a risk factor with diseases that have been considered to be caused by environmental factors at a relatively high level (e.g., diabetes or hypertension). It is becoming evident that many of them are defined by single nucleotide polymorphisms. Accordingly, the analysis of SNPs is considered to lead to medication or therapy that is compatible with individual constitutions (tailor-made medication). This has drawn much attention.

Up to the present, a variety of methods for the analysis of SNPs have been developed. These methods are roughly classified into two types: a method for detecting unknown nucleotide substitution and a method for detecting known nucleotide substitution.

Examples of methods for analyzing known nucleotide substitution include a method of directly analyzing the nucleotide sequence and a method of employing a DNA chip with oligonucleotide.

With respect to genetic examination for tailor-made medication, a technology for typing SNPs of patients associated with each disease will be required in the future. In addition, more effective therapy can be expected though the determination of guidelines for medication or therapy as rapidly as possible. Therefore, development of a technique for typing SNPs, which can be simply carried out by anybody and which can quickly yield results, is desired.

At present methods for typing know SNPs are theoretically classified into two methods; i.e., a method of utilizing polymerase reactions and a method of utilizing hybridization.

There are three types of methods of utilizing hybridization, i.e., the simple hybridization method utilizing a DNA chip (sequence by hybridization, Drmanac R. et al.: Genomics 4: 114-128 (1989)), the dye-labeled oligonucleotide ligation method (Chen X. et al.: Genome Res. 8: 549-556 (1998)), and the invader method (Lyamichev et al.: Science 260: 778-783 (1993)). In any case, an oligonucleotide corresponding to each allele is prepared, and it is detected which allele the oligonucleotide is hybridized with.

Disadvantageously, these methods are time-consuming due to the necessity for hybridization, and the necessary apparatuses are expensive due to the use of fluorescence in the detection system. Thus, examination cannot be simply carried out.

Methods of utilizing polymerase reactions are classified into two types. One of them is a method in which a primer is set close to SNP to determine which nucleotide was incorporated at the SNP site. Examples thereof are the SNaPShot method and the Pyrosequence method (Alderborn, A. et al.: Genome Res., 28: 1249-1258 (2000)). Another type is a method in which a primer is designed to contain the SNP site corresponding to each allele around the 3′ terminus, and SNPs are determined based on the occurrence of polymerase reactions. Examples thereof are the amplification refractory mutation system (ARMS) method (Newton C R, et al.: Nucl Acids Res. 17: 2503-2516 (1989)) and the PCR-amplification of specific alleles (PASA) method (Sarker G et al.: Anal Biochem 186: 64-68 (1990)).

In the SNaPShot method, a primer is provided in such a manner that it reaches a position immediately before the SNP site, elongation is carried out using dideoxynucleotide only, and the nucleotide that is incorporated is then analyzed. Since this process is the elongation of only one nucleotide, a sequencer must be used for analysis. Accordingly, to is a disadvantage in that the use of an expensive apparatus is required.

In the Pyrosequence method, a primer is located several nucleotides upstream or immediately downstream the SNP, and, starting therefrom, a sequence reaction is carried out with the addition of one deoxynucleotide. In this case, pyrophosphoric acid, which is generated only upon elongation, is converted to ATP to generate chemiluminescence, and this luminescence is detected. Since the amount of pyrophosphoric acid generated is proportional to the number of nucleotides incorporated, this method is excellent in terms of quantitativeness. This method, however, is problematic with respect to the cost and the operability, since four types of dNTPs must be added to the reaction site, and an apparatus for detecting luminescence is required.

The ARMS method or the PASA method utilizes the characteristics of the reaction in that elongation staring from the primer is strongly dependent on the level of match between the 3′ terminus of the primer and a template (Kwok S. et al.: Nucleic Acids Res 18, 999-1005 (1990), Huang M. M. et al.: Nucleic Acids Res. 20, 4567-4573 (1992)). More specifically, in this method, a primer that is complementary to each allele is previously prepared, and the genotype is determined based on the occurrence of amplification utilizing characteristics, i.e., the fact that elongation occurs only when the primer is congruous with the genotype of the sample. This method is excellent due to the possibility of quick inspection by a simple method such as electrophoresis.

In fact however, there is only one nucleotide difference between each allele-specific primer, and non-specific amplification often occurs with the mismatched primers depending on the template sequence (Huang M. M. et al.: Nucleic Acids Res. 20, 4567-4573 (1992)). It is difficult to prevent non-specific amplification since subtle conditions, such as the choice of the apparatus to be used or the surrounding environment affect the occurrence of amplification.

In the ARMS method, another mismatch is artificially introduced into a position located one nucleotide upstream of the 3′ terminus of the primer for the purpose of enhancing the level of mismatch. This is performed because the introduction of two mismatched nucleotides within 4 nucleotides from the 3′ terminus can significantly lower the amplification efficiency of PCR (Kwok S. et al.: Nucleic Acids Res 18, 999-1005 (1990)). Thus, non-specific amplification can be prevented to some extent without strictly regulating conditions.

Nucleotide polymorphisms seem to be clearly detectable by the aforementioned methods. However, if different primers are used in the amplification reaction such as PCR, efficiency of amplification derived from both primers can be often different. If the amounts of the amplification products corresponding to both alleles are not the same, the amounts of the amplified products of both alleles are different in the case where the SNP type is heterozygous. Thus, it become difficult to distinguish a heterozygote from a homozygote.

### DISCLOSURE OF THE INVENTION

An object of the preset invention is to provide a method for accurately, simply, and quickly detecting single nucleotide polymorphisms. More particularly, another object of the present invention is to provide a method for clearly distinguishing the heterozygous allele from the homozygous allele.

The present inventors have conducted concentrated studies in order to attain the above object. As a result, they have found that the amplified product of a heterozygous allele can be distinguished from that of a homozygous allele by designing allele-specific primers in such a way that the amounts of the amplification products derived from each heterozygous allele are substantially the same, and using such two types of allele-specific primers.

More specifically, primers specific to both alleles were designed to contain the SNP sites and to contain artificial mismatches in the vicinity of the SNP site to enhance the level of mismatch. In this case, artificially mismatched nucleotides are selected in such a way that the levels of mismatch between an each allele of a heterozygous sample and a primer corresponding thereto are the same levels. This makes the amounts of amplification products of each allele the same. Thus, a heterozygous allele can be distinguished from a homozygous allele.

Further, the present inventors have found that the amplified product of a heterozygous allele can be distinguished from that of a homozygous allele by utilizing each of two types of allele-specific primers under such different polymerase reaction conditions that the amounts of the amplified products of each of heterozygous alleles are substantially the same.

More specifically, they have succeeded in making the amounts of the amplified products of each of alleles substantially the same by respectively setting PCR conditions for each of allele-specific primers. Thus, a heterozygous allele can be distinguished from a homozygous allele by setting PCR conditions respectively.

The present invention provides a method for detecting single nucleotide polymorphisms, which utilizes two types of allele-specific primers designed in such a way that the amounts of the amplified products of each of heterozygous alleles are substantially the same.

According to another aspect the present invention provides a method for detecting single nucleotide polymorphisms which utilizes two types of allele-specific primers under such polymerase reaction conditions that the amounts of the amplified products of each of heterozygous alleles are substantially the same.

Preferably, the allele-specific primer is designed to have a polymorphic site within 4 nucleotides from the 3′ terminus of the allele-specific primer.

Preferably, the allele-specific primer comprises a mismatched nucleotide introduced to the nucleotide adjacent to the polymorphism site.

Preferably, the allele-specific primer comprises a mismatched nucleotide adjacent to the polymorphic site which is selected for each allele.

Preferably, single nucleotide polymorphisms are detected by utilizing polymerase reactions.

Preferably, the polymerase reaction is PCR reaction.

Preferably, the amplified products of each of heterozygous alleles becomes substantially the same by using different number of reaction cycles for each allele-specific primer in the PCR using two types of allele-specific primers.

Preferably, the amplified products of each of heterozygous alleles becomes substantially the same by using different primer concentrations for each allele-specific primer in the PCR using two types of allele-specific primers.

Preferably, the amplified products of each of heterozygous alleles becomes substantially the same by using different initial amounts of a template for each allele-specific primer in the PCR using two types of allele-specific primers.

Preferably, single nucleotide polymorphisms are detected by using a product of polymerase reactions.

Preferably, single nucleotide polymorphisms are detected by employing electrophoresis, chromatography or HPLC as a detection means.

Preferably, single nucleotide polymorphisms are detected using a by-product of polymerase reactions.

Preferably, the by-product is pyrophosphoric acid.

Preferably, pyrophosphoric acid is detected using a dry analytical element.

Preferably, the detection of single nucleotide polymorphisms comprises determining homo/heterozygosity of single nucleotide polymorphisms.

According to further another aspect, the present invention provides a primer set for carrying out the method according to the present invention, which comprises two types of allele-specific primers designed in such a way that the amounts of the amplified products of each allele are substantially the same.

### BRIEF DESCRIPTION OF THE DRAWING

FIG. 1 is a diagram showing a concept of the embodiment of the present invention.

### EMBODIMENTS FOR CARRYING OUT THE INVENTION

The embodiments of the present invention are described in detail below.

(1) Primer Design

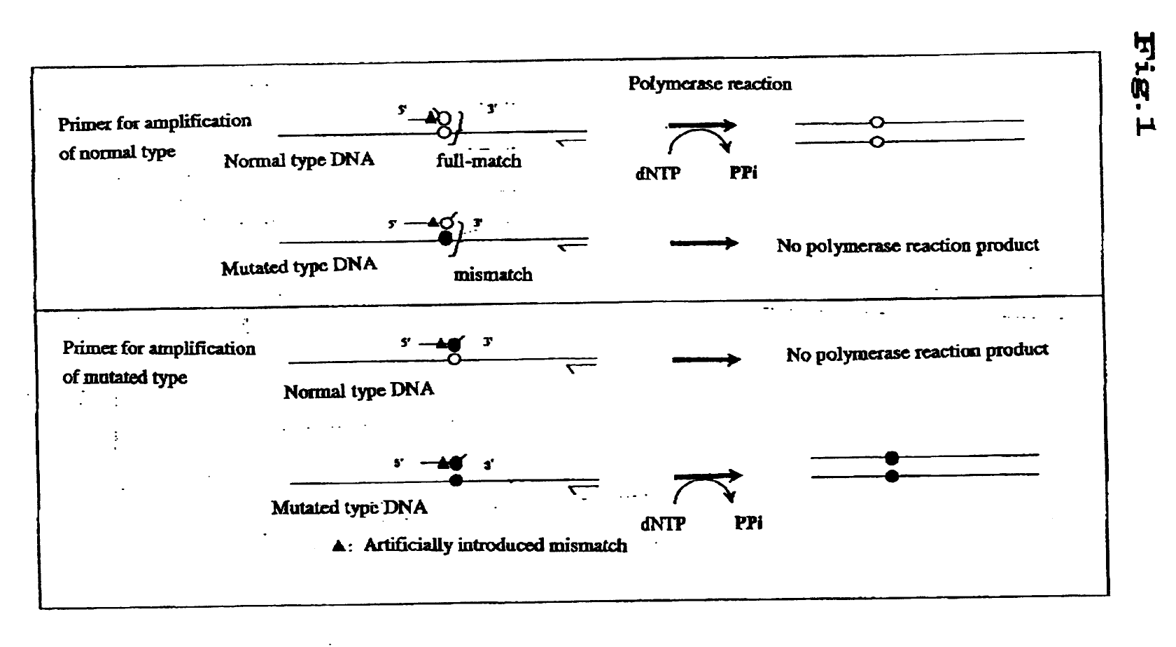

The first method for detecting single nucleotide polymorphisms according to the present invention is characterized in the use of two types of allele-specific primers designed in such a way that the amounts of the amplified products of each of heterozygous alleles are substantially the same. By utilizing the method according to the present invention, homo/heterozygosity of the single nucleotide polymorphisms can be determined. In a preferable embodiment of the method according to the present invention, polymerase reaction is carried out using a primer specific to each allele, and the occurrence of elongation reaction is detected. Specific examples of the methods for detection include methods for directly assaying amplified products such as electrophoresis, mass analysis or liquid chromatography, and a method for detecting pyrophosphoric acid generated upon the polymerase elongation. FIG. 1 is a diagram showing a concept of the embodiment of the present invention.

The preferable embodiment of the method for determining the homo/heterozygosity of single nucleotide polymorphisms according to the present invention is described below,

An allele-specific primer is designed in such a way that an SNP site to be detected is contained. In this case, mismatch is artificially introduced in the vicinity of the SNP site in order to enhance the level of mismatch to an allele. Artificial mismatch is selected in such a way that the level of mismatch between each allele and each primer is the same. These primers are separately used to conduct polymerase elongation.

Whether or not elongation actually occurs is determined by the detection of pyrophosphoric acid. More preferably, pyrophosphoric acid is detected by using a dry analytical element for pyrophosphoric acid quantification which comprises a reagent layer containing xanthosine or inosine, pyrophosphatase, purine nucleoside phosphorylase, xanthine oxidase peroxidase, and a color developer.

(2) Polymerase Reaction Condition

The second method for detecting single nucleotide polymorphisms according to the present invention is characterized in the use of two types of allele-specific primers under such polymerase reaction conditions that the amounts of the amplified products of each of heterozygous alleles are substantially the same. More specifically, when the polymerase reaction is PCR and the PCR is carried out using two types of allele-specific primers, it is possible to make the amounts of the amplified products of each of heterozygous alleles substantially the same (i) by using different number of reaction cycles for each allele-specific primer, (ii) by using different primer concentrations for each allele-specific primer and/or (iii) using different initial amounts of a template for each allele-specific primer. By utilizing the method according to the present invention, homo/heterozygosity of the single nucleotide polymorphisms can be determined. In a preferable embodiment of the method according to the present invention, polymerase reaction is carried out using a primer specific to each allele, and the occurrence of elongation reaction is detected. Specific examples of the methods for detection include methods for directly assaying amplified products such as electrophoresis, mass analysis or liquid chromatography, and a method for detecting pyrophosphoric acid generated upon the polymerase elongation. FIG. 1 is a diagram showing a concept of the embodiment of the present invention.

The preferable embodiment of the method for determining the homo/heterozygosity of single nucleotide polymorphisms according to the present invention is described below.

An allele-specific primer is designed in such a way that an SNP site to be detected is contained. In this case, mismatch may be artificially introduced in the vicinity of the SNP site in order to enhance the level of mismatch to an allele, or alternatively such artificial mismatch may not be introduced. These primers are separately used to conduct polymerase elongation.

Whether or not elongation actually occurs is determined by the detection of pyrophosphoric acid. More preferably, pyrophosphoric acid is detected by using a dry analytical element for pyrophosphoric acid quantification which comprises a reagent layer containing xanthosine or inosine, pyrophosphatase, purine nucleoside phosphorylase, xanthine oxidase, peroxidase, and a color developer.

The embodiments of the present invention are hereafter described in more detail

(A) Target Nucleic Acid Fragment:

A target nucleic acid fragment to be analyzed in the present invention is polynucleotide, where at least a part of its nucleotide sequence is known. The target nucleic acid fragment can be a genomic DNA fragment isolated from any organism including an animal, microorganism, bacterium, and plant. Also, a cDNA fragment, which is synthesized using an RNA fragment or DNA fragment isolatable from viruses and mRNA as a template, can be analyzed. Preferably, the target nucleic acid fragment is purified to as great an extent as possible, and extra ingredients other than the nucleic acid fragment are removed. For example, when a genomic DNA fragment isolated from the blood of an animal (e.g., a human) or when nucleic acid (DNA or RNA) fragments of infectious bacteria or viruses in blood are analyzed, leucocyte membranes destroyed in the isolation process, hemoglobin eluted from erythocytes, and other general chemical substances in blood should be fully removed. In particular, hemoglobin inhibits the subsequent polymerase elongation.

(B) Primer Specific to Target Nucleic Acid Fragment:

A primer specific to a target nucleic acid fragment used in the present invention is an oligonucleotide having a nucleotide sequence complementary to a site of interest where the nucleotide sequence of the target nucleic acid fragment is known. Hybridization of the primer complementary to the target nucleic acid fragment to the site of interest on the target nucleic acid fragment results in progress in polymerase elongation starting from the 3′ terminus of the primer and using the target nucleic acid as a template. More specifically, whether the primer recognizes and specifically hybridizes to the site of interest on the target nucleic acid fragment or not is an important issue for the present invention. The number of nucleotides in the primer used in the present invention is preferably 5 to 60, and particularly preferably 15 to 40.

Further, the primer used in the present invention should be designed in such a way that the polymorphic site, at least one of which is intended to be detected, is contained. More preferably, the polymorphic site is provided within 4 nucleotides from the 3′ terminus. This is necessary because the detection of polymorphisms according to the present invention utilizes the characteristics of the reaction in that elongation starting from the primer is strongly dependent on the level of mismatch between the 3′ terminus of the primer and a template. If mismatch is present in the vicinity of the 3′ terminus, elongation dose not proceed. In fact, however, if there is only one nucleotide difference at the SNP site to be detected between allele-specific primers, non-specific amplification can occur even with the use of mismatched primers depending on the template sequence. Since subtle conditions such as the choice of the apparatus to be used or the surrounding environment affect the occurrence of amplification, it is difficult to prevent non-specific amplification. Thus, polymorphisms cannot be determined. Accordingly, mismatch can be artificially introduced to a position in addition to the SNP polymorphic sites. Artificial mismatch is preferably located in the vicinity of the SNP polymorphic site, and it is more preferably located adjacent thereto. This can prevent non-specific amplification to some extent.

However, if different primers are used in the amplification reaction such as PCR, the efficiency of amplification derived from both primers can be often different.

In the case of heterozygous alleles, the amounts of amplified products of both alleles are different from each other. This could result in mistake judgment as being homozygous alleles. Accordingly, in the first aspect of the present invention, artificial mismatch is provided in such a way that the amounts of amplified products from both alleles become equal. For example, when a certain gene has the SNP of (G/A), a sequence of the primer for detecting the G type may be designed to be . . . AG . . . , and a sequence of the primer for detecting the A type may be designed to be . . . CA . . . . Further, in the second aspect of the invention, PCR conditions are set in such a way that the amounts of amplified products from both alleles become equal. In the present invention, the amounts of the amplified products of each of heterozygous alleles can be substantially the same (i) by using different number of reaction cycles for each allele-specific primer, (ii) by using different primer concentrations for each allele-specific primer and/or (iii) using different initial amounts of a template for each allele-specific primer.

(C) Polymerase:

When the target nucleic acid is DNA, the polymerase used in the present invention is DNA polymerase, which catalyzes complementary elongation staring from the double-strand region formed by hybridization of the primer to the region of the target nucleic acid fragment which was denatured into a single stand in the 5′→3′ direction, using deoxynucleoside triphosphate (dNTP) as a component, and using the target nucleic acid fragment as a template. Specific examples of DNA polymerase which can be used include DNA polymerase I, Klenow fragment of DNA polymerase I, and Bst DNA polymerase. DNA polymerase can be selected or combined depending on the purpose. For example, when a part of the target nucleic acid fragment is amplified (e.g., via PCR), use of Taq DNA polymerase, which is excellent in terms of heat resistance, is effective. Use of DNA polymerase α, T4 DNA polymerase, and T7 DNA polymerase which have hexokinase activity in the 3′→5′ direction in combination is also possible.

When a genomic nucleic acid or mRNA of RNA viruses is the target nucleic acid fragment, reverse transcriptase having reverse transcription activity can be used. Further, reverse transcriptase can be used in combination with Taq DNA polymerase.

(D) Polymerase Elongation Reaction:

Polymerase elongation reaction in the present invention includes all the elongation processes of complementary nucleic acids. These elongation processes proceed by staring from the 3′ terminus of a primer complementary to the target nucleic acid fragment as described in (B) above, which was specifically hybridized to a part of the region of the target nucleic acid fragment which was denatured into a single strand as described in (A). Also, deoxynucleoside triphosphates (dNTP) are used as components, the polymerase as described in (C) above is used as a catalyst, and the target nucleic acid fragment is used as a template. This elongation reaction of complementary nucleic acids indicates that continuous elongation reaction occurs at least twice (corresponding to 2 nucleotides).

When the amount of the target nucleic acid is small, a site of interest in the target nucleic acid is preferably amplified by any means utilizing polymerase elongation reaction. In the amplification of the target nucleic acid, various methods that have been heretofore invented and developed can be used.

Examples of methods for amplification of nucleic acid include PCR (JP Patent Publication (Kokoku) Nos. 4-67960 B (1992) and 4-67957 B (1992)), LCR (JP Patent Publication (Kokai) No. 5-2934 A (1993)), stand displacement amplification (SDA, JP Patent Publication (Kokai) No. 5-130870 A (1993)), rolling circle amplification (RCA, Proc, Natl. Acad. Sci vol. 92, 4641-4645 (1995)), isothermal and chimeric primer-initiated amplification of nucleic acids (ICAN), loop-mediated isothermal amplification of DNA (LAMP, Bio Industry, vol. 18, No. 2, (2001)), the nucleic acid sequence-based amplification (NASBA) method (Nature 350, 91 (1991)), and the transcription mediated amplification (TMA) method (J. Clin Microbiol., vol. 31, 3270 (1993)).

The most general and widespread method for amplifying the target nucleic acid is polymerase chain reaction (PCR). PCR is a method for amplifying a site of interest on the target nucleic acid fragment by repeating periodical processes of denaturing (a step of denaturing a nucleic acid fragment from double-stranded to single-stranded)→annealing (a step of hybridizing a primer to a nucleic acid fragment which was denatured into a single strand)→polymerase (Taq DNA polymerase) elongation reaction→denaturing, by the periodical control of increase and decease in temperature of the reaction solution. Finally, the site of interest on the target nucleic acid fragment can be amplified 1,000,000 times compared to the initial amount.

When the target nucleic acid fragment is an RNA fragment, elongation reaction can be carried out using the RNA strand as a template by using reverse transcriptase having reverse transcription activity. Further, RT-PCR can be carried out by using reverse transcriptase in combination with Taq DNA polymerase and performing reverse transcription (RT), followed by PCR.

In LCR (JP Patent Publication (Kokai) No. 5-2934 A (1993)), two complementary oligonucleotide probe strands are bound to a single-stranded DNA by end-to-tail, and a nick between two oligonucleotide strands is sealed with a heat-resistant ligase. The bound DNA strands are released by denaturation, become a template, and arm then amplified. Preparation of probe sequence with some contrivance enables SNP determination based on the occurrence of amplification. Also, a method improved from LCR has been also developed in which a gap is provided between two primers and this gap is filled with polymerase (Gap-LCR: Nucleic Acids Research, vol. 23, No. 4, 675 (1995)). Utilization of this method enables SNP determination by the assay of pyrophosphoric acid which is generated upon the polymerase elongation.

Strand displacement amplification (SDA, JP Patent Publication (Kokai) No. 5-130870 A (1993)) is a cycling assay method using exonuclease, which is a method for amplifying a site of interest on the target nucleic acid fragment by utilizing polymerase elongation reaction. This method is a method of decomposing a primer from a reverse direction by performing polymerase elongation starting from a primer specifically hybridized with a site of interest on the target nucleic acid fragments and also allowing 5′→3′ exonuclease to act. In place of the decomposed primer, a new primer is hybridized, and elongation reaction by DNA polymerase proceeds again. This elongation reaction by polymerase and decomposition reaction by exonuclease for removing the previously elongated strand are successively and periodically repeated. Elongation reaction by polymerase and decomposition reaction by exonuclease can be carried out under isothermal conditions. Preparation of a primer sequence with some contrivance enables SNP determination based on the occurrence of polymerase reaction.

The LAMP method is a recently developed method for amplifying a site of interest on a target nucleic acid fragment. This method is carried out by using at least 4 types of primer's, which complimentarily recognize at least 6 specific sites of the target nucleic acid fragment, and strand displacement-type Bst DNA polymerase which has no nuclease activity in the 5′→3′ direction and which catalyzes elongation reaction while allowing the double-stranded DNA on the template to be released as single-stranded DNA, and a site of interest on the target nucleic acid fragment is amplified as a special structure under isothermal conditions. Preparation of a primer sequence with some contrivance enables SNP determination based on the occurrence of amplification. The amplification efficiency of the LAMP method is high, and the amount of accumulated pyrophosphoric acid generated upon polymerase elongation reaction is very large. This facilitates the SNP detection by the detection of pyrophosphoric acid.

The ICAN method is also a recently developed method for amplifying a site of interest on the target nucleic acid fragment. This is a method for gene amplification under isothermal conditions utilizing an RNA-DNA chimeric primer, DNA polymerase having a stand displacement activity and a template exchange activity, and RNaseH. After the chimeric primer is bound to a template, a complementary strand is synthesized by DNA polymerase. Thereafter, RNaseH cleaves the RNA portion derived from the chimeric primer, and elongation reaction which involves strand displacement reaction and template exchange reaction occurs from the cleaved site. This procedure is repeated several times, and thus, the gene is amplified. Preparation of a primer sequence with some contrivance enables SNP determination based on the occurrence of amplification. The amplification efficiency of the ICAN method is high, and the amount of accumulated pyrophosphoric acid generated upon polymerase elongation reaction is very large. This facilitates the SNP detection by the detection of pyrophosphoric acid.

(E) Detection:

An object of the present invention is to make the amounts of products after the polymerase reactions such as PCR the same between the products derived from heterozygous alleles. Accordingly, methods for detection are not limited as long as the amounts of the products can be quantified.

Examples of methods for detection include methods for directly assaying the amount of the generated products such as electrophoresis, liquid chromatography or mass analysis, and a method for detecting pyrophosphoric acid or the like generated upon the polymerase reaction. With respect to quantitativeness, a detection method by quantification of pyrophosphoric acid is preferable. With respect to simplicity, a method for quantifying pyrophosphoric acid using a dry analytical element is preferable.

A method represented by formula 1 has been heretofore known as a method for detecting pyrophosphoric acid (PPi). In this method, pyrophosphoric acid (PPi) is converted into adenosinetriphosphate (ATP) with the aid of sulfurylase, and luminescence generated when adenosinetriphosphate acts on luciferin with the aid of luciferase is detected. Thus, an apparatus capable of measuring luminescence is required for detecting pyrophosphoric acid (PPi) by this method.

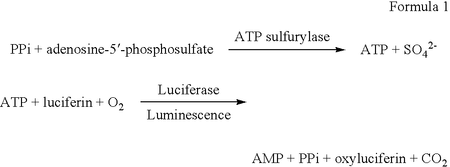

A method for detecting pyrophosphoric acid suitable for the present invention is a method represented by formula 2 or 3. In the method represented by formula 2 or 3, pyrophosphoric acid (PPi) is converted into inorganic phosphate (Pi) with the aid of pyrophosphatase, inorganic phosphate (Pi) is reacted with xanthosine or inosine with the aid of purine nucleoside phosphorylase (PNP), the resulting xanthine or hypoxanthine is oxidated with the aid of xanthine oxidase (XOD) to generate uric acid, and a color developer (a dye precursor) is allowed to develop color with the aid of peroxidase (POD) using hydrogen peroxide (H₂O₂) generated in the oxidation process, followed by colorimetry. In the method represented by formula 2 or 3, the result can be detected by colorimetry and, thus, pyrophosphoric acid (PPi) can be detected visually or using a simple colorimetric measuring apparatus.

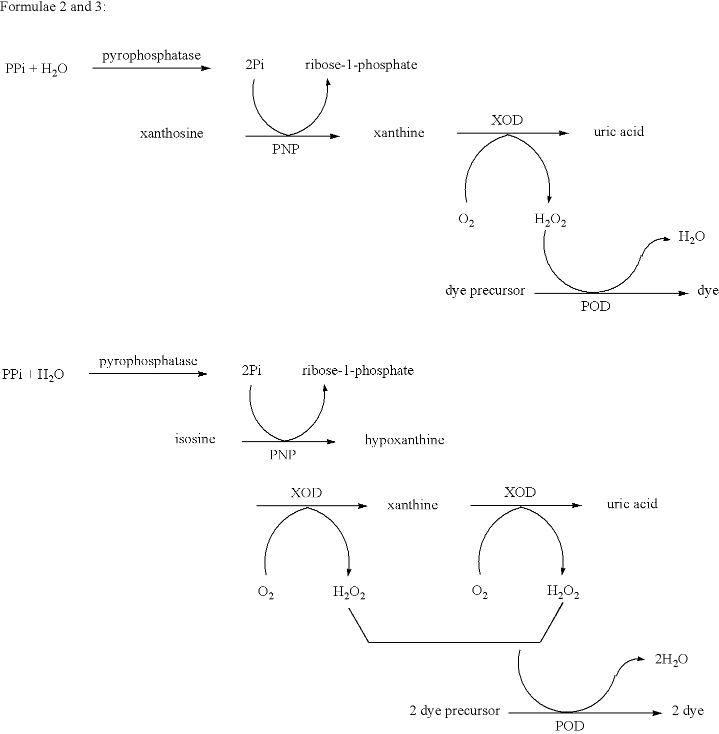

Commercially available pyrophosphatase (EC3, 6, 1, 1), purine nucleoside phosphorylase (PNP, EC2. 4.2. 1), xanthine oxidase (XOD, EC1. 2. 3. 2), and peroxidase (POD, EC1, 11. 1. 7) can be used. A color developer (i.e., a dye precursor) may be any one as long as it can generate a dye by hydrogen peroxide and peroxidase (POD), and examples thereof which can be used herein include: a composition which generates a dye upon oxidation of leuco dye (e.g., triarylimidazole leuco dye described in U.S. Pat. No. 4,089,747 and the like, diarylimidazole leuco dye described in Japanese Patent Publication Laying-Open No. 59-193352 (EP 0122641A)); and a composition (e.g., 4-aminoantipyrines and phenols or naphthols) containing a compound generating a dye by coupling with other compound upon oxidation.

(F) Dry Analytical Element:

A dry analytical element which can be used in the present invention is an analytical element which comprises a single or a plurality of functional layers, wherein at least one layer (or a plurality of layers) comprises a detection reagent, and a dye generated upon reaction in the layer is subjected to quantification by colorimetry by reflected light or transmitted light from the outside of the analytical element.

In order to perform quantitative analysis using such a dry analytical element, a given amount of liquid sample is spotted onto the surface of a developing layer. The liquid sample spread on the developing layer reaches the reagent layer and reacts with the reagent thereon and develops color. After spotting, the dry analytical element is maintained for a suitable period of time at given temperature (for incubation) and a color developing reaction is allowed to thoroughly proceed. Thereafter, the reagent layer is irradiated with an illuminating light from, for example, a transparent support side, the amount of reflected light in a specific wavelength region is measured to determine the optical density of reflection, and quantitative analysis is carried out based on the previously determined calibration curve.

Since a dry analytical element is stored and kept in a dry state before detection, it is not necessary that a reagent is prepared for each use. As stability of the reagent is generally higher in a dry state, it is better than a so-called wet process in terms of simplicity and swiftness since the wet process requires the preparation of the reagent solution for each use. It is also excellent as an examination method because highly accurate examination can be swiftly carried out with a very small amount of liquid sample.

(G) Dry Analytical Element for Pyrophosphoric Acid Quantification:

A dry analytical element for quantifying pyrophosphoric acid which can be used in the present invention can have a layer construction which is similar to various known dry analytical elements. The dry analytical element may be multiple layers which contain, in addition to a reagent for performing the reaction represented by formula 2 or 3 according to item (E) above (detection of pyrophosphoric acid (PPi)), a support, a developing layer, a detection layer, a light-shielding layer, an adhesive layer, a water-absorption layer, an undercoating layer, and other layers. Examples of such dry analytical elements include those disclosed in the specifications of Japanese Patent Publication Laying-Open No. 49-53888 (U.S. Pat. No. 3,992,158), Japanese Patent Publication Laying-Open No. 51-40191 (U.S. Pat. No. 4,042,335), Japanese Patent Publication Laying-Open No. 55-164356 (U.S. Pat. No. 4,292,272), and Japanese Patent Publication Laying-Open No. 61-4959 (EPC Publication No. 0166365A).

Examples of the dry analytical element to be used in the present invention include a dry analytical element for quantitative assay of pyrophosphoric acid which has a reagent layer comprising a reagent which converts pyrophosphoric acid into inorganic phosphorus, and a group of reagents capable of color reaction depending on the amount of inorganic phosphorus.

In this dry analytical element for quantitative assay of pyrophosphate, pyrophosphoric acid (PPi) can enzymatically be converted into inorganic phosphorus (Pi) using pyrophosphatase as described above. The subsequent process, that is color reaction depending on the amount of inorganic phosphorus (Pi), can be performed using “quantitative assay method of inorganic phosphorus” (and combinations of individual reactions used therefor), described hereinafter, which is known in the field of biochemical inspection.

It is noted that when representing “inorganic phosphorus,” both the expressions “Pi” and “HPO₄²⁻, H₂PO₄¹⁻” are used for phosphoric acid phosphate ion). Although the expression “Pi” is used in the examples of reactions described below, the expression “HPO₄²⁻” may be used for the same reaction formula.

As the quantitative assay method of inorganic phosphorus, an enzyme method and a phosphomolybdate method are known. Hereinafter, this enzyme method and phosphomolybdate method will be described as the quantitative assay method of inorganic phosphorus.

A. The Enzyme Method

Depending on the enzyme to be used for the last color reaction during a series of reactions for Pi quantitative detection, the following methods for quantitative assay are available: using peroxidase (POD); or using glucose-6-phosphate dehydrogenase (G6PDH), respectively. Hereinafter, examples of these methods are described.

(1) Example of the Method Using Peroxidase (POD)

(1-1)

Inorganic phosphorus (Pi) is allowed to react with inosine by purine nucleoside phosphorylase (PNP), and the resultant hypoxanthine is oxidized by xanthine oxidase (XOD) to produce uric acid. During this oxidization process, hydrogen peroxide (H₂O₂) is produced. Using the thus produced hydrogen peroxide, 4-aminoantipyrines (4-AA) and phenols are subjected to oxidization-condensation by peroxidase (POD) to form a quinoimine dye, which is colorimetrically assessed.

(1-2)

Pyruvic acid is oxidized by pyruvic oxidase (POP) in the presence of inorganic phosphorus (Pi), cocarboxylase (TPP), flavin adenine dinucleotide (FAD) and Mg²⁺ to produce acetyl acetate. During this oxidization process, hydrogen peroxide (H₂O₂) is produced. Using the thus produced hydrogen peroxide, 4-aminoantipyrines (4-AA) and phenols are subjected to oxidization-condensation by perxidase (POD) to form a quinonimine dye which is colorimetrically assessed, in the same manner as described in (1-1).

It is noted that the last color reaction for each of the above processes (1-1) and (1-2) can be performed by a “Trinder reagent” which is known as a detection reagent for hydrogen peroxide. In this reaction, phenols function as “hydrogen donors.” Phenols to be used as “hydrogen donors” are classical, and now various modified “hydrogen donors” are used. Examples of these hydrogen donors include N-ethyl-N-sulfopropyl-m-anilidine, N-ethyl-N-sulfopropylaniline, N-ethyl-N-sulfopropyl-3,5-dimethoxyaniline, N-sulfopropyl-3,5-dimethoxyamiline, N-ethyl-N-sulfopropyl-3,5-dimethylaniline, N-ethyl-N-sulfopropyl-m-toluidine, N-ethyl-N-(2-hydroxy-3-sulfopropyl)-m-anilidine, N-ethyl-N-(2-hydroxy-3-sulfopropyl)aniline, N-ethyl-N-(2-hydroxy-3-sulfopropyl)-3,5-dimethoxyaniline, N-(2-hydroxy-3-sulfopropyl)-3,5-dimethoxyaniline, N-ethyl-N-(2-hydroxy-3-sulfopropyl)-3,5-dimethylaniline, N-ethyl-N-(2-hydroxy-3-sulfopropyl)-m-toluidine, and N-sulfopropylaniline.

(2) Methods Using glucose-6-phosphate dehydrogenase (G6PDH)

(2-1)

Inorganic phosphorus (Pi) is reacted with glycogen with phosphorylase to produce glucose-1-phosphate (G1-P). The produced glucose-1-phosphate is converted into glucose-6-phosphate (G-6-P) with phosphoglucomutase (PGM). In the presence of glucose-6-phosphate and nicotiamide adenine dinucleotide (NAD), NAD is reduced to NADH with glucose-6-phosphate dehydrogenase (G6PDH), followed by colorimetric analysis of the produced NADH.

(2-2)

Inorganic phosphorus (Pi) is reacted with maltose with maltose phosphorylase (MP) to produce glucose-1-phosphate (G-1-P). Thereafter, the produced glucose-1-phosphate is converted into glucose-6-phosphate (G-6-P) with phosphoglucomutase (PGM) in the same manner as described in (2-1). In the presence of glucose-6-phosphate and nicotiamide adenine dinucleotide (NAD), NAD is reduced to NADH with glucose-6-phosphate dehydrogenase (G6PDH), followed by colorimetric analysis of the produced NADH.

B. Phosphomolybdate Method

There are two phosphomolybdate methods. One is a direct method wherein “Phosphomolybdates (H₃[PO₄Mo₁₂O₃₆])” prepared by complexing inorganic phosphorus (phosphate) and aqueous molybdate ions under acidic condition are directly quantified. The other is a reduction method wherein further to the above direct method, Mo(IV) is reduced to Mo(III) by a reducing agent and molybudenum blue (Mo(III)) is quantified. Examples of the aqueous molybdate ions include aluminum molybdate, cadmium molybdate, calcium molybdate, barium molybdate, lithium molybdate, potassium molybdate, sodium molybdate, and ammonium molybdate. Representative examples of the reducing agents to be used in the reduction method include 1-amino-2-naphthol-4-sulfonic acid, ammonium ferrous sulfate, ferrous chloride, stannous chloride-hydrazine, p-methylaminophenol sulfate, N,N-dimethyl-phenylenediamine, ascorbic acid, and malachite green.

When a light-transmissive and water-impervious support is used, the dry analytical element can be practically constructed as below. However, the scope of the present invention is not limited to these.

(1) One having a reagent layer on the support.

(2) One having a detection layer and a reagent layer in that order on the support.

(3) One having a detection layer, a light reflection layer, and a reagent layer in that order on the support.

(4) One having a second reagent layer, a light reflection layer, and a first reagent layer in that order on the support.

(5) One having a detection layer, a second reagent layer, a light reflection layer, and a first reagent layer in that order on the support.

In (1) to (3) above, the reagent layer may be constituted by a plurality of different layers. For example, a first reagent layer may contain enzyme pyrophosphatase which is required in the pyrophosphatase reaction represented by formula 2 or 3, and substrate xanthosine or substrate inosine and enzyme PNP which are required in the PNP reaction, a second reagent layer may contain enzyme XOD which is required in the XOD reaction represented by formula 2 or 3, and a third reagent layer may contain enzyme POD which is required in the POD reaction represented by formula 2 or 3, and a coloring dye (dye precursor). Alternatively, two reagent layers are provided. On the first reagent layer, the pyrophosphatase reaction and the PNP reaction may be proceeded, and the XOD reaction and the POD reaction may be proceeded on the second reagent layer. Alternatively, the pyrophosphatase reaction, the PNP reaction and the XOD reaction may be proceeded on the first reagent layer, and the POD reaction may be proceeded on the second reagent layer.

A water absorption layer may be provided between a support and a reagent layer or detection layer. A filter layer may be provided between each layer. A developing layer may be provided on the reagent layer and an adhesive layer may be provided therebetween.

Any of light-nontransmissive (opaque), light-semitransmissive (translucent), or light-transmissive (transparent) support can be used. In general, a light-transmissive and water-impervious support is preferred. Preferable materials for a light-transmissive and water-impervious support are polyethylene terephthalate or polystyrene. In order to firmly adhere a hydrophilic layer, an undercoating layer is generally provided or hydrophilization is carried out.

When a porous layer is used as a reagent layer, the porous medium may be a fibrous or nonfibrous substance. Fibrous substances used herein include, for example, filter paper, non-woven fabric, textile fabric (e.g. plain-woven fabric), knitted fabric (e.g., tricot knitted fabric), and glass fiber filter paper. Nonfibrous substances may be any of a membrane filter comprising cellulose acetate etc., described in Japanese Patent Publication Laying-Open No. 49-53888 and the like, or a particulate structure having mutually interconnected spaces comprising fine particles of inorganic substances or organic substances described in, for example, Japanese Patent Publication Laying-Open No. 49-53888, Japanese Patent Publication Laying-Open No. 55-90859 (U.S. Pat. No. 4,258,001), and Japanese Patent Publication Lying-Open No. 58-70163 (U.S. Pat. No. 4,486,537). A partially-adhered laminate which comprises a plurality of porous layers described in, for example, Japanese Patent Publication Laying-Open No. 61-4959 (EP Publication 0166365A), Japanese Patent Publication Laying-Open No. 62-116258, Japanese Patent Publication Laying-Open No. 62-138756 (EP Publication 0226465A), Japanese Patent Publication Laying-Open No. 62-138757 (EP Publication 0226465A), and Japanese Patent Publication Laying-Open No, 62-138758 (EP Publication 0226465A), is also preferred.

A porous layer may be a developing layer having so-called measuring action, which spreads liquid in an area substantially in proportion to the amount of the liquid to be supplied. Preferably, a developing layer is textile fabric, knitted fabric, and the like. Textile fabrics and the like may be subjected to glow discharge treatment as described in Japanese Patent Publication Laying-Open No. 57-66359. A developing layer may comprise hydrophilic polymers or surfactants as described in Japanese Patent Publication Laying-Open No. 60-222770 (EP 0162301A), Japanese Patent Publication Laying-Open No. 63-219397 (German Publication DE 3717913A), Japanese Patent Publication Laying-Open No. 63-112999 (DE 3717913A), and Japanese Patent Publication Laying-Open No. 62-182652 (DE 3717913A) in order to regulate a developing area, a developing speed and the like.

For example, a method is useful where the reagent of the present invention is previously impregnated into or coated on a porous membrane etc., comprising paper, fabric or polymer, followed by adhesion onto another water-pervious layer provided on a support (e.g., a detection layer) by the method as described in Japanese Patent Publication Laying-Open No. 55-1645356.

The thickness of the reagent layer thus prepared is not particularly limited. When it is provided as a coating layer, the thickness is suitably in the range of about 1 μm to 50 μm, preferably in the range of 2 μm to 30 μm. When the reagent layer is provided by a method other than coating, such as lamination, the thickness can be significantly varied in the range of several tens of to several hundred μm.

When a reagent layer is constituted by a water-pervious layer of hydrophilic polymer binders, examples of hydrophilic polymers which can be used include: gelatin and a derivative thereof (e.g., phthalated gelatin); a cellulose derivative (e.g., hydroxyethyl cellulose); agarose, sodium arginate; an acrylamide copolymer or a methacrylamide copolymer (e.g., a copolymer of acrylamide or methacrylamide and various vinyl monomers); polyhydroxyethyl methacrylate; polyvinyl alcohol; polyvinyl pyrrolidone; sodium polyacrylate; and a copolymer of acrylic acid and various vinyl monomers.

A reagent layer composed of hydrophilic polymer binders can be provided by coating an aqueous solution or water dispersion containing the reagent composition of the present invention and hydrophilic polymers on the support or another layer such as a detection layer followed by drying the coating in accordance with the methods described in the specifications of Japanese Patent Examined Publication No. 53-21677 (U.S. Pat. No. 3,992,158), Japanese Patent Publication Laying-Open No. 55-164356 (U.S. Pat. No. 4,292,272), Japanese Patent Publication Laying-Open No. 54-101398 (U.S. Pat. No. 4,132,528) and the like. The thickness of the reagent layer comprising hydrophilic polymers as binders is about 2 μm to about 50 μm, preferably about 4 μm to about 30 μm on a dry basis, and the coverage is about 2 g/m² to about 50 g/m², preferably about 4 g/m² to about 30 g/m².

The reagent layer can further comprise an enzyme activator, a coenzyme, a surfactant, a pH buffer composition, an impalpable powder, an antioxidant, and various additives comprising organic or inorganic substances in addition to the reagent composition represented by formula 2 or 3 in order to improve coating properties and other various properties of diffusible compounds such as diffusibility, reactivity, and storage properties. Examples of buffers which can be contained in the reagent layer include pH buffer systems described in “Kagaku Binran Kiso (Handbook on Chemistry, Basic),” The Chemical Society of Japan (ed.), Maruzen Co., Ltd. (1996), p.1312-1320, “Data for Biochemical Research, Second Edition, R. M. C. Dawson et al. (2^{nd} ed.), Oxford at the Clarendon Press (1969), p. 476-508, “Biochemistry” 5, p. 467-477 (1966), and “Analytical Biochemistry” 104, p. 300-310 (1980). Specific examples of pH buffer systems include a buffer containing borate; a buffer containing citric acid or citrate, a buffer containing glycine, a buffer containing bicine; a buffer containing HEPES; and Good's buffers such as a buffer containing MES. A buffer containing phosphate cannot be used for a dry analytical element for detecting pyrophosphoric acid.

The dry analytical element for quantifying pyrophosphoric acid which can be used in the present invention can be prepared in accordance with a known method disclosed in the above-described various patent specifications. The dry analytical element for quantifying pyrophosphoric acid is cut into small fragments, such as, an about 5 mm to about 30 mm-square or a circle having substantially the same size, accommodated in the slide frame described in, for example, Japanese Patent Examined Publication No. 57-283331 (U.S. Pat. No. 4,169,751), Japanese Utility Model Publication Laying-Open No. 56-142454 (U.S. Pat. No. 4,387,990), Japanese Patent Publication Laying-Open No. 57-63452, Japanese Utility Model Publication Laying-Open No. 58-32350, and Japanese Patent Publication Laying-Open No. 58-501144 (International Publication WO 083/00391), and used as slides for chemical analysis. This is preferable from the viewpoints of production, packaging, transportation, storage, measuring operation, and the like. Depending on its intended use, the analyical element can be accommodated as a long tape in a cassette or magazine, as small pieces accommodated in a container having an opening, as small pieces applied onto or accommodated in an open card, or as small pieces cut to be used in that state.

The dry analytical element for quantifying pyrophosphoric acid which can be used in the present invention can quantitatively detect pyrophosphoric acid which is a test substance in a liquid sample, by operations similar to that described in the above-described patent specifications and the like. For example, about 2 μL to about 30 μL preferably 4 μL to 15 μL of aqueous liquid sample solution is spotted on the reagent layer. The spotted analytical element is incubated at constant temperature of about 20° C. to about 45° C., preferably about 30° C. to about 40° C. for 1 to 10 minutes. Coloring or discoloration in the analytical element is measured by the reflection from the light-transmissive support side, and the amount of pyrophosphoric acid in the specimen can be determined based on the principle of colorimetry using the previously prepared calibration curve. Quantitative analysis can be carried out with high accuracy by keeping the amount of liquid sample to be spotted, the incubation time, and the temperate at constant levels.

Quantitative analysis can be carried out with high accuracy in a very simple operation using chemical analyzers described in, for example, Japanese Patent Publication Laying-Open No. 60-125543, Japanese Patent Publication Laying-Open No. 60-220862, Japanese Patent Publication Laying-Open No. 61-294367, and Japanese Patent Publication Laying-Open No. 58-161867 (U.S. Pat. No. 4,424,191). Semiquantitative measurement may be carried out by visually judging the level of coloring depending on the purpose and accuracy needed.

Since the dry analytical element for quantifying pyrophosphoric acid which can be used in the present invention is stored and kept in a dry state before analysis, it is not necessary that a reagent is prepared for each use, and stability of the reagent is generally higher in a dry state. Thus, in terms of simplicity and swiftness, it is better than a so-called wet process, which requires the preparation of the reagent solution for each use. It is also excellent as an examination method because highly accurate examination can be swiftly carried out with a very small amount of liquid sample.

The dry analytical element for quantifying inorganic phosphorus which can be used in the second aspect of the present invention can be prepared by removing pyrophosphatase from the reagent layer in the aforementioned dry analytical element for quantifying pyrophosphoric acid. The dry analytical element described in Japanese Patent Publication Laying-Open No. 7-197 can also be used. The dry analytical element for quantifying inorganic phosphors is similar to the aforementioned dry analytical element for quantifying pyrophosphoric acid in its layer construction, method of production, and method of application, with the exception that the reagent layer does not comprise pyrophosphatase.

The present invention is described in more detail with reference to the examples, but the technical scope of the present invention is not limited by these examples.

### EXAMPLES

### Reference Example 1 (Comparative Example)

Detection of single nucleotide polymorphisms (SNPs) of aldehyde dehydrogenase gene (ALDH2 gene)-associated site using a dry analytical element for pyrophosphoric acid quantification (an example in which a site corresponding to single nucleotide polymorphisms is set around the 3′ terminus of the primer)

(1) Preparation of Sample Solution of Nucleic Acid Containing Target Nucleic Acid Fragment

From each of the blood specimens collected from subjects, who are previously known to have either of the active, less-active or inactive form of ALDH2 by nucleotide sequencing, genomic nucleic acid fragments were extracted and purified by using a commercially available kit for extracting and purifying nucleic acid (QIAGEN, QLAamp DNA Blood Mini Kit). The active, less-active or inactive form of ALDH2 occurs due to differences in a specific type of nucleotide in the ALDH2 gene-associated site. The resultants DNA fragments were collected in 1 mL of purified distilled water to prepare a sample solution of nucleic acid containing the target nucleic acid fragment.

(2) Preparation of Dry Analytical Element for Pyrophosphoric Acid Quantification

A colorless transparent polyethylene terephthalate (PET) smooth film sheet (support) comprising a gelatin undercoating layer (thickness of 180 μm) was coated with an aqueous solution having composition (a) shown in Table 1 at the following coverage. The coating was then dried to provide a reagent layer.

TABLE 1
Composition (a) of aqueous solution for reagent layer

| | | |
|---|---|---|
| Gelatin | 18.8 | g/m² |
| p-Nonylphenoxy polyxydol | 1.5 | g/m² |
| (glycidol unit: containing 10 on average) | | |
| (C₉H₁₉—Ph—O—(CH₂CH(OH)—CH₂—O)₁₀H) | | |
| Xanthosine | 1.96 | g/m² |
| Peroxidase | 15,000 | IU/m² |
| Xanthine oxidase | 13,600 | IU/m² |
| Purine nucleoside phosphorylase | 3,400 | IU/m² |
| Leuco dye | 0.28 | g/m² |
| (2-(3,5-dimethoxy-4-hydroxyphenyl)-4- | | |
| phenethyl-5-(4-dimethylaminophenyl)imidazole) | | |
| Water | 136 | g/m² |

(pH was adjusted to 6.8 with a diluted NaOH solution)

This reagent layer was coated with an aqueous solution for an adhesive layer having composition (b) shown in Table 2 below at the following coverage. The coating was then dried to provide an adhesive layer.

TABLE 2
Composition (b) of aqueous solution for adhesive layer

| | |
|---|---|
| Gelatin | 3.1 g/m² |
| p-Nonylphenoxy polyxydol | 0.25 g/m² |
| (glycidol unit: containing 10 on average) | |
| (C₉H₁₉—Ph—O—(CH₂—CH(OH)—CH₂—O)₁₀H) | |
| Water | 59 g/m² |

Subsequently, water was supplied to the adhesive layer over its whole surface at 30 g/m² to allow the gelatin layer to swell. A broad textile fabric made of genuine polyester was laminated thereon by applying slight pressure in a substantially even manner to provide a porous developing layer.

The developing layer was then substantially evenly coated with an aqueous solution having composition (c) shown in Table 3 below at the following coverage. The coating was then dried, cut into a size of 13 mm×14 mm, and accommodated into a plastic mounting material, thereby preparing a dry analytical element for pyrophosphoric acid quantification.

TABLE 3
Composition (c) of aqueous solution for developing layer

| | | |
|---|---|---|
| HEPES | 2.3 | g/m² |
| Sucrose | 5.0 | g/m² |
| Hydroxypropyl methylcellulose | 0.04 | g/m² |
| (methoxy group 19% to 24%, | | |
| hydroxypropoxy group 4% to 12%) | | |
| Pyrophosphatase | 14,000 | IU/m² |
| Water | 98.6 | g/m² |

(pH was adjusted to 7.2 with a diluted NaOH solution)

(3) Amplification by PCR

Sample solutions of nucleic acid containing the target nucleic acid fragments, which were obtained in (1) above by extracting and purifying from human whole blood samples either having the active or inactive form of ALDH2, were used as they were, and amplification by PCR was carried out under the following conditions.

<Primer>

A primer (upper) common in the ALDH2 gene-associated site on chromosome 12 and a set of two primes were used. These two primers are a primer (lower-1) and a primer (lower-2) corresponding to the active and the inactive forms of ALDH2 in which a portion corresponding to single nucleotide polymorphisms that determines the ALDH2 activity is set around the 3′-terminus (underlined portion of the primer nucleotide sequence described in lower-1 and lower-2). Mismatch was artificially produced by changing a nucleotide (T→A) which is located one nucleotide upstream on the 5′ side in the sequence corresponding to single nucleotide polymorphisms of the lower primer.

Primers for detecting active form

| | |
|---|---|
| Primers for detecting active form | |
| Primer (upper): | |
| 5′-AACGAAGCCCAGCAAATGA-3′ | (SEQ ID NO:1) |
| Primer (lower-1): | |
| 5′-GGGCTGCAGGCATACACAGA-3′ | (SEQ ID NO:2) |
| Primers for detecting inactive form | |
| Primer (upper): | |
| 5′-AACGAAGCCCAGCAAATGA-3′ | (SEQ ID NO:1) |
| Primer (lower-2): | |
| 5′-GGGCTGCAGGCATACACAAA-3′ | (SEQ ID NO:3) |

Amplification by PCR was carried out using a reaction solution having the composition below by repeating 35 cycles of denaturing at 94° C. for 20 seconds, annealing at 60° C. for 30 seconds, and polymerase elongation at 72° C. for 1 minute and 30 seconds.

<Composition of reaction solution>

| | |
|---|---|
| 10 × PCR buffer | 5 μL |
| 2.5 mM dNTP | 5 μL |
| 5 μM primer (upper) | 2 μL |
| 5 μM primer (lower-1 or lower-2) | 2 μL |
| Taq | 0.5 μL |
| Sample solution of nucleic acid fragment obtained in (1) | 0.5 μL |
| Purified water | 35 μL |

(4) Detection Using an Analytical Element for Pyrophosphoric Acid Quantification

20 μL of each of the solutions after amplification by PCR in (3) above were spotted as they were on the dry analytical element for pyrophosphoric acid quantification prepared in (2) above, and the dry analytical elements for pyrophosphoric acid quantification were incubated at 37° C. for 5 minutes. Thereafter, the optical density of reflection (ODR) was measured at the wavelength of 650 nm from the support side. The obtained results are shown in Table 4 below.

TABLE 4

| | Active primer | Inactive primer |
|---|---|---|
| Active form | 0.517 | 0.449 |
| Less-active form | 0.545 | 0.501 |
| Inactive form | 0.471 | 0.505 |

According to the results of Reference Example 1, the forms of ALDH-2 in the sample which have been known have a consistent relationship with the optical density of reflection (ODR) measured using a dry analytical element for pyrophosphoric acid quantification. This indicates that single nucleotide polymorphisms (SNPs) of an aldehyde dehydrogenase gene (ALDH2 gene)-associated site can be detected. Since the amounts of pyrophosphoric acids generated are different between less-active alleles, it is difficult to distinguish an active form from a less-active form.

### Example 1

Detection of single nucleotide polymorphisms (SNPs) of aldehyde dehydrogenase gene (ALDH2 gene)-associated site using a dry analytical element for pyrophosphoric acid quantification (an example in which a nucleotide of a primer is set depending on each allele)

(1) Preparation of Sample Solution of Nucleic Acid Containing Target Nucleic Acid Fragment

In the same manner as in Reference Example 1, sample solutions of nucleic acid containing target nucleic acid fragment were prepared from blood preparations of each of subjects, who were known to have the active, less-active or inactive form of ALDH2 by nucleotide sequencing.

(2) Preparation of a Dry Analytical Element for Pyrophosphoric Acid Quantification

The dry analytical element was prepared in the same manner as in Reference Example 1.

(3) Amplification by PCR

Sample solutions of nucleic acid containing the target nucleic acid fragments, which were obtained in (1) above by extracting and purifying from human whole blood samples either having the active or inactive form of ALDH2, were used as they were, and amplification by PCR was carried out under the following conditions.

<Primer>

A primer (upper) common in the ALDH2 gene-associated site on chromosome 12 and a set of two primers were used. These two primers are a primer (lower-1) and a primer (lower-2) corresponding to the active and the inactive forms of ALDH2 in which a portion corresponding to single nucleotide polymorphisms that determines the ALDH2 activity is set around the 3′-terminus (underlined portion of the primer nucleotide sequence described in lower-1 and lower-2). Mismatch was artificially produced by changing a nucleotide (T→A or C→A) which is located one nucleotide upstream on the 5′ side in the sequence corresponding to single nucleotide polymorphisms of the lower primer.

| | |
|---|---|
| <Primers for detecting active form> | |
| Primer (upper): | |
| 5′-AACGAAGCCCAGCAAATGA-3′ | (SEQ ID NO:1) |
| Primer (lower-1A): | |
| 5′-GGGCTGCAGGCATACACAGA-3′ | (SEQ ID NO:4) |
| Primer (lower-1C): | |
| 5′-GGGCTGCAGGCATACACCGA-3 | (SEQ ID NO:5) |
| <Primers for detecting inactive form> | |
| Primer (upper): | |
| 5′-AACGAAGCCCAGCAAATGA-3′ | (SEQ ID NO:1) |
| Primer (lower-2A): | |
| 5′-GGGCTGCAGGCATACACAAA-3′ | (SEQ ID NO:6) |
| Primer (lower-2C): | |
| 5′-GGGCTGCAGGCATACACCAA-3′ | (SEQ ID NO:7) |

Amplification by PCR was carried out using a reaction solution having the composition below by repeating 35 cycles of denaturing at 94° C. for 20 second annealing at 60° C. for 30 seconds, and polymerase elongation at 72° C. for 1 minute and 30 seconds.

<Composition of reaction solution>

| | |
|---|---|
| 10 × PCR buffer | 5 μL |
| 2.5 mM dNTP | 5 μL |
| 5 μM primer (upper) | 2 μL |
| 5 μM primer (lower-1A and 1C or lower-2A and 2C) | 2 μL |
| Taq | 0.5 μL |
| Sample solution of nucleic acid fragment obtained in (1) | 0.5 μL |
| Purified water | 35 μL |

(4) Detection Using Analytical Element for Pyrophosphoric Acid Quantification

20 μl of each of the solutions after amplification by PCR in (3) above were spotted as they were on the dry analytical elements for pyrophosphoric acid quantification prepared in (2) above, and the dry analytical elements for pyrophosphoric acid quantification were incubated at 37° C. for 5 minutes. Thereafter, the optical density of reflection (ODR) was measured at the wavelength of 650 nm from the support side. The obtained results are shown in Table 5.

TABLE 5

| | |
|---|---|
| Active | Inactive |
| primer | primer |

| | 1A | 1C | 2A | 2C |
|---|---|---|---|---|
| Active | 0.517 | 0.547 | 0.449 | 0.457 |
| Less-active | 0.545 | 0.548 | 0.505 | 0.544 |
| Inactive | 0.471 | 0.474 | 0.501 | 0.565 |

According to the results of Example 1, the optical densities of reflection (ODR) measured by using a dry analytical element for pyrophosphoric acid quantification are equivalent to each other in a less-active sample by the use of the primer 1A as an active primer and the primer 2C as an inactive primer. This enabled the discrimination of an active/inactive form from a less-active form.

### Example 2

Detection of single nucleotide polymorphisms (SNPs) of aldehyde dehydrogenase gene (ALDH2 gene)-associated site using a dry analytical element for pyrophosphoric acid quantification (an example in which PCR conditions are set depending on each allele)

(1) Preparation of Sample Solution of Nucleic Acid Containing Target Nucleic Acid Fragment

In the same manner as in Reference Example 1, sample solutions of nucleic acid containing target nucleic acid fragment were prepared from blood preparations of a subject, who was known to have the less-active form of ALDH2 by nucleotide sequencing.

(2) Preparation of a Dry Analytical Element for Pyrophosphoric Acid Quantification

The dry analytical element was prepared in the same manner as in Reference Example 1.

(3) Amplification by PCR

Sample solutions of nucleic acid containing the target nucleic acid fragments, which were obtained in (1) above by extracting and purifying from human whole blood samples either having the less active form of ALDH2, were used as they were, and amplification by PCR was carried out under the following conditions.

<Primer>

A primer (upper) common in the ALDH2 gene-associated site on chromosome 12 and a set of two primers were used. These two primers are a primer lower-1) and a primer (lower-2) corresponding to the active and the inactive forms of ALDH2 in which a portion corresponding to single nucleotide polymorphisms that determines the ALDH2 activity is set around the 3′-terminus (underlined portion of the primer nucleotide sequence described in lower-1 and lower-2). Mismatch was artificially produced by changing a nucleotide (T→A) which is located one nucleotide upstream on the 5′ side in the sequence corresponding to single nucleotide polymorphisms of the lower primer.

Primers for detecting active form

| | |
|---|---|
| Primers for detecting active form | |
| Primer (upper): | |
| 5′-AACGAAGCCCAGCAAATGA-3′ | (SEQ ID NO:1) |
| Primer (lower-1): | |
| 5′-GGGCTGCAGGCATACACAGA-3′ | (SEQ ID NO:2) |
| Primers for detecting inactive form | |
| Primer (upper): | |
| 5′-AACGAAGCCCAGCAAATGA-3′ | (SEQ ID NO:1) |
| Primer (lower-2): | |
| 5′-GGGCTGCAGGCATACACAAA-3′ | (SEQ ID NO:3) |

Amplification by PCR was carried out using a reaction solution having the composition below by repeating 33 cycles of denaturing at 94° C. for 20 seconds, annealing at 60° C. for 30 seconds, and polymerase elongation at 72° C. for 1 minute and 30 seconds, for the primers for detecting active form, or by repeating 35 cycles of the same as above for the primers for detecting inactive form.

<Composition of reaction solution>

| | |
|---|---|
| 10 × PCR buffer | 5 μL |
| 2.5 mM dNTP | 5 μL |
| 5 μM primer (upper) | 2 μL |
| 5 μM primer (lower-1 or lower-2) | 2 μL |
| Taq | 0.5 μL |
| Sample solution of nucleic acid fragment obtained in (1) | 0.5 μL |
| Purified water | 35 μL |

(4) Detection Using Analytical Element for Pyrophosphoric Acid Quantification

20 μl of each of the solutions after amplification by PCR in (3) above were spotted as they were on the dry analytical elements for pyrophosphoric acid quantification prepared in (2) above, and the dry analytical elements for pyrophosphoric acid quantification were incubated at 37° C. for 5 minutes. Thereafter, the optical density of reflection (ODR) was measured at the wavelength of 650 nm from the support side. The obtained results are shown in Table 6.

TABLE 6

| | Active type | Inactive type |
|---|---|---|
| | primer (33 cycles) | primer (35 cycles) |
| Less-active form | 0.530 | 0.529 |

According to the results of Example 2, the optical densities of reflection (ODR) measured by using a dry analytical element for pyrophosphoric acid quantification are equivalent to each other in a less-active sample by changing the PCR conditions (in this case, the number of the cycle) for the active type primer and the inactive type primer. This enabled the discrimination of an active/inactive form from a less-active form.

### EFFECT OF THE INVENTION

The present invention enables accurate, simple, and quick detection of single nucleotide polymorphisms. More particularly, the present invention enables clear distinction between a heterozygous allele and a homozygous allele.

The contents of Japanese Patent Applications Nos.2002-289566 and 2002-289567, which the present application claims priorities based on, are incorporated herein by reference as a part of the disclosure of the present application.

What is claimed is:

1. A method for detecting single nucleotide polymorphisms, which utilizes two types of allele-specific primers designed in such a way that the amounts of the amplified products of each of heterozygous alleles are substantially the same.

2. The method according to claim 1 wherein the allele-specific primer is designed to have a polymorphic site within 4 nucleotides from the 3′ terminus of the allele-specific primes.

3. The method according to claim 1 wherein the allele-specific primer comprises a mismatched nucleotide introduced to the nucleotide adjacent to the polymorphism site.

4. The method according to claim 1 wherein the allele-specific primer comprises a mismatched nucleotide adjacent to the polymorphic site which is selected for each allele.

5. The method according to claim 1 wherein single nucleotide polymorphisms are detected by utilizing polymerase reactions.

6. The method according to claim 1 wherein single nucleotide polymorphisms are detected by using a product of polymerase reactions.

7. The method according to claim 6 wherein single nucleotide polymorphisms are detected by employing electrophoresis, chromatography or HPLC as a detection means.

8. The method according to claim 1 wherein single nucleotide polymorphisms are detected using a by-product of polymerase reactions.

9. The method according to claim 8 wherein the by-product is pyrophosphoric acid.

10. The method according to claim 9 wherein pyrophosphoric acid is detected using a dry analytical element.

11. The method according to claim 1 wherein the detection of single nucleotide polymorphisms comprises determining homo/heterozygosity of single nucleotide polymorphisms.

12. A primer set for carrying out the method according to claim 1, which comprises two types of allele-specific primers designed in such a way that the amounts of the amplified products of each allele are substantially the same.

13. A method for detecting single nucleotide polymorphisms, which utilizes two types of allele-specific primers under such polymerase reaction conditions that the amounts of the amplified products of each of heterozygous alleles are substantially the same.

14. The method according to claim 13 wherein the polymerase reaction is PCR reaction.

15. The method according to claim 13 wherein the amplified products of each of heterozygous alleles becomes substantially the same by using different number of reaction cycles for each allele-specific primer in the PCR using two types of allele-specific primers.

16. The method according to claim 13 wherein the amplified products of each of heterozygous alleles becomes substantially the same by using different primer concentrations for each allele-specific primer in the PCR using two types of allele-specific primers.

17. The method according to claim 13 wherein the amplified products of each of heterozygous alleles becomes substantially the same by using different initial amounts of a template for each allele-specific primer in the PCR using two types of allele-specific primers.

18. The method according to claim 13 wherein the allele-specific primer is designed to have a polymorphic site within 4 nucleotides from the 3′ terminus of the allele-specific primer.

19. The method according to claim 13 wherein single nucleotide polymorphisms are detected by using a product of polymerase reactions.

20. The method according to claim 19 wherein single nucleotide polymorphisms are detected by employing electrophoresis, chromatography or HPLC as a detection means.

21. The method according to claim 13 wherein single nucleotide polymorphisms are detected using a by-product of polymerase reactions.

22. The method according to claim 21 wherein the by-product is pyrophosphoric acid.

23. The method according to claim 13 wherein pyrophosphoric acid is detected using a dry analytical element.

24. The method according to claim 13 wherein the detection of single nucleotide polymorphisms comprises determining homo/heterozygosity of single nucleotide polymorphisms.
